(19) United States
(12) Patent Application Publication
(10) Pub. No.: US 2026/0158016 A1
(43) Pub. Date: Jun. 11, 2026

(54) HALOPERIDOL DERIVATIVES, PHARMACEUTICAL COMPOSITION COMPRISING SAID DERIVATIVES, AND THERAPEUTIC USES THEREOF

(71) Applicant: University of Virginia Patent Foundation, Charlottesville, VA (US)

(72) Inventors: Jayakrishna AMBATI, Charlottesville, VA (US); Vidya Lakshmi AMBATI, Charlottesville, VA (US); Praveen YERRAMOTHU, Charlottesville, VA (US); Yosuke NAGASAKA, Charlottesville, VA (US); Bradley David GELFAND, Charlottesville, VA (US); Kameshwari AMBATI, Charlottesville, VA (US); Brian C. WERNER, Charlottesville, VA (US); Shao-Bin WANG, Charlottesville, VA (US)

(21) Appl. No.: 19/150,122

(22) PCT Filed: Feb. 2, 2024
(86) PCT No.: PCT/US2024/014186

§ 371 Date: Jul. 22, 2025

Related U.S. Application Data
(60) Provisional application No. 63/483,029, filed on Feb. 3, 2023.
(60) Provisional application No. 63/606,708, filed on Dec. 6, 2023.

(51) Int. Cl. A61K 31/451; A61P 27/02

(52) U.S. Cl. CPC A61K 31/451; A61P 27/02

(57) ABSTRACT

The present disclosure provides for methods of use of the haloperidol and haloperidol derivatives and their pharmaceutical compositions to treat eye pathologies. In an aspect, the eye pathology can be geographic atrophy, dry macular degeneration, wet macular degeneration, choroidal neovascularization, diabetic retinopathy, diabetic macular edema, retinal vein occlusion, neovascular glaucoma, retinal detachment, proliferative vitreoretinopathy, or glaucoma. The administration of the treatment can be via the eye.

### CROSS-REFERENCE TO RELATED APPLICATION

This application claims priority to U.S. provisional application entitled “HALOPERIDOL INHIBITS INFLAMMASOME ACTIVATION AND RETINAL PIGMENTED EPITHELIUM DEGENERATION” having Ser. No. 63/483,029 filed on Feb. 3, 2023, and this application claims priority to U.S. provisional application entitled “HALOPERIDOL DERIVATIVES, PHARMACEUTICAL COMPOSITION COMPRISING SAID DERIVATIVES, THERAPEUTIC USE THEREOF” having Ser. No. 63/606,708 filed on Dec. 6, 2023, both of which are entirely incorporated herein by reference.

### STATEMENT ON FUNDING PROVIDED BY THE U.S. GOVERNMENT

This invention was made with Government support under contract R01EY031039 awarded by the National Institutes of Health. The Government has certain rights in the invention.

### BACKGROUND

Age-related macular degeneration (AMD) affects over 200 million people globally (Wong, 2014). Geographic atrophy, a late stage of the “dry” form of AMD that is characterized by degeneration of the retinal pigmented epithelium (RPE) (Ambati, 2003). This vision-threatening condition affects over 5 million around the world and has no U.S. Food and Drug Administration (FDA)-approved treatment.

### SUMMARY

The present disclosure provides for methods of use of the haloperidol and haloperidol derivatives and their pharmaceutical compositions to treat eye pathologies.

The present disclosure provides for a method for treating an eye pathology comprising: administering to a subject in need thereof, a pharmaceutical composition, wherein the pharmaceutical composition comprises a therapeutically effective amount of the compound or the pharmaceutical composition, wherein the compound has a formula represented by structure I or the pharmaceutically acceptable salt thereof:

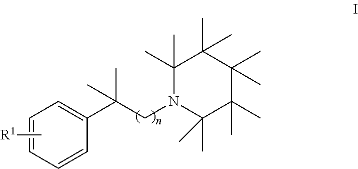

wherein
each R¹ is independently selected from hydrogen, halogen, and a C1 to C6 alkyl group;
R^{2a} and R^{2b} are each independently selected from hydrogen or oxygen, when R^{2a} is oxygen, oxygen has a double bond to the carbon atom and the carbon atom is not bonded to R^{2b} due to the double bond to carbon;
n is an integer from 0 to 8; and
R^{3a1}, R^{3a2}, R^{3b1}, R^{3b2}, R^{3c1}, R^{3c2}, R^{3d1}, R^{3d2}, R^{3e1} and R^{3e2} are independently selected from hydrogen, halogen, —OH, a C1 to C6 alkyl group, and a substituted or unsubstituted aryl group.

In an aspect, the method includes treating one of the following eye pathologies is one of: geographic atrophy, dry macular degeneration, wet macular degeneration, choroidal neovascularization, diabetic retinopathy, diabetic macular edema, retinal vein occlusion, neovascular glaucoma, retinal detachment, proliferative vitreoretinopathy, or glaucoma.

In an aspect, the method includes ocular administration. In one aspect, ocular administration is selected from one of: intravitreous administration, suprachoroidal administration, sub-Tenon administration, periocular administration, subretinal administration, intracameral administration, or topical administration.

### BRIEF DESCRIPTION OF DRAWINGS

Further aspects of the present disclosure will be more readily appreciated upon review of the detailed description of its various embodiments, described below, when taken in conjunction with the accompanying drawings.

FIG. 1 illustrates Kaplan-Meier survival plot showing the probability of not developing dry AMD (survival) over time for subjects in the PearlDiver Mariner database based on haloperidol (Haldol) exposure or nonexposure. Dotted lines show the 95% confidence interval. Difference between haloperidol exposure or nonexposure groups was significant (<2×10⁻¹⁶ by log-rank test).

FIG. 2 illustrates that haloperidol inhibits inflammasome activation. ELISA-based quantification of cytokine release show that haloperidol (1-10 μM) inhibits IL-1β secretion by THP-1 cells induced by LPS and ATP. n=3 per group. Mean and SEM values are presented. P<0.05 for each of the haloperidol-treated groups compared to vehicle-treated group, following LPS+ATP stimulation, Student's t test.

FIG. 3 illustrates Alu RNA-induced RPE degeneration is blocked by haloperidol. FIG. 3A illustrates rates of protection against RPE degeneration induced by subretinal administration of Alu RNA for various doses of intravitreously-administered haloperidol. FIG. 3B illustrates RPE flat mounts stained for zonula occludens-1 (ZO-1; red) following subretinal administration of Alu RNA and intraperitoneal administration of either vehicle or various doses of haloperidol. Loss of regular hexagonal cellular boundaries in ZO-1-stained flat mounts is indicative of degenerated RPE.

### DETAILED DESCRIPTION

In general, the present disclosure provides for treating eye pathologies such as age-related macular degeneration using haloperidol derivatives and the like. Additional details are provided herein and in the Examples.

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated.

### Definitions

It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

It will be understood by those skilled in the art that the moieties substituted can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “alkyl”, either alone or within other terms such as “thioalkyl” and “arylalkyl”, as used herein, means a monovalent, saturated hydrocarbon radical which may be a straight chain (i.e. linear) or a branched chain. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like. An alkyl radical for use in the present disclosure generally comprises from about 1 to 20 carbon atoms, particularly from about 1 to 10, 1 to 8 or 1 to 7, more particularly about 1 to 6 carbon atoms, or 3 to 6. Illustrative alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-actyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain aspects of the disclosure an alkyl radical is a C₁-C₆ lower alkyl comprising or selected from the group comprising methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents as defined herein at positions that do not significantly interfere with the preparation of compounds of the disclosure and do not significantly reduce the efficacy of the compounds. In certain aspects of the disclosure, an alkyl radical is substituted with one to five substituents including halo, lower alkoxy, lower aliphatic, a substituted lower aliphatic, hydroxy, cyano, nitro, thio, amino, keto, aldehyde, ester, amide, substituted amino, carboxyl, sulfonyl, sulfuryl, sulfenyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, cycloaliphatic, substituted cycloaliphatic, or aryl (e.g., phenylmethyl benzyl)), heteroaryl (e.g., pyridyl), and heterocyclic (e.g., piperidinyl, morpholinyl). Substituents on an alkyl group may themselves be substituted.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkenyl” as used herein is a hydrocarbon group of from 2 to 24 carbon atoms or 2 to 8 carbon atoms or 2 to 6 carbon atoms with a structural formula containing at least one carbon-carbon double bond. Asymmetric structures such as (R¹R²)C═C(R³R⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

As used herein, “alkynyl” or “alkynyl group” refers to straight or branched chain hydrocarbon groups having 2 to 40, 2 to 20, 2 to 10, or 2 to 5 carbon atoms and at least one triple carbon to carbon bond, such as ethynyl. Reference to “alkynyl” or “alkynyl group” includes unsubstituted and substituted forms of the hydrocarbon moiety.

The term “cycloalkynyl” as used herein is a non-aromatic carbon-based ring composed of at least seven carbon atoms and containing at least one carbon-carbon triple bound. Examples of cycloalkynyl groups include, but are not limited to, cyclooctynyl, cyclononynyl, and the like. The term “heterocycloalkynyl” is a type of cycloalkenyl group as defined above and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The terms “alkoxyl” or “alkoxyalkyl” as used herein refer to an alkyl-O— group wherein alkyl is as described herein. The term “alkoxyl” as used herein can refer to C1-20 inclusive, linear, branched, or cyclic, saturated or unsaturated oxo-hydrocarbon chains, including, for example, methoxyl, ethoxyl, propoxyl, isopropoxyl, n-butoxyl, t-butoxyl, and pentoxyl.

The Ar (e.g., Ar₁, Ar₂, etc) group is an aromatic system or group such as an aryl group. “Aryl”, as used herein, refers to C₅-C₂₀-membered aromatic, heterocyclic, fused aromatic, fused heterocyclic, biaromatic, or bihetereocyclic ring systems. In an aspect, “aryl”, can include 5-, 6-, 7-, 8-, 9-, and 10-membered single-ring aromatic groups that may include from zero to four heteroatoms, for example, functional groups that correspond to benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems (C₅-C₃₀) having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

In some aspects, a structure of a compound can be represented by a formula:

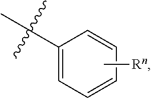

which is understood to be equivalent to a formula:

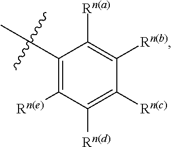

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

The term “carboxyl” as used herein, alone or in combination, refers to —C(O)OR₂₅— or —C(—O)OR₂₅ wherein R₂₅ is hydrogen, alkyl, alkenyl, alkynyl, cycloalkyl, cycloalkenyl, amino, thiol, aryl, heteroaryl, thioalkyl, thioaryl, thioalkoxy, a heteroaryl, or a heterocyclic, which may optionally be substituted. In aspects of the disclosure, the carboxyl groups are in an esterified form and may contain as an esterifying group lower alkyl groups. In particular aspects of the disclosure, —C(O)OR₂₅ provides an ester or an amino acid derivative. An esterified form is also particularly referred to herein as a “carboxylic ester”. In aspects of the disclosure a “carboxyl” may be substituted, in particular substituted with allyl which is optionally substituted with one or more of amino, amine, halo, alkylamino, aryl, carboxyl, or a heterocyclic. Examples of carboxyl groups are methoxycarbonyl, butoxycarbonyl, tert.alkoxycarbonyl such as tert-butoxycarbonyl, arylmethyoxycarbonyl having one or two aryl radicals including without limitation phenyl optionally substituted by for example lower alkyl, lower alkoxy, hydroxyl, halo, and/or nitro, such as benzyloxycarbonyl, methoxybenzyloxycarbonyl, diphenylmethoxycarbonyl, 2-bromoethoxycarbonyl, 2-iodoethoxycarbonyltert.butylcarborlyl, 4-nitrobenzyloxycarbonyl, diphenylmethoxy-carbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine). Examples of silicon substituents include trimethylsilyi and dimethyltertbutylsilyl. In aspects of the disclosure, the carboxyl group may be an alkoxy carbonyl, in particular methoxy carbonyl, ethoxy carbonyl, isopropoxy carbonyl, t-butoxycarbonyl, t-pentyloxycarbonyl, sir heptyloxy carbonyl, especially methoxy carbonyl or ethoxy carbonyl.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, but not intended to be limiting, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

A composition of the disclosure can be a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the disclosure, e.g. encapsulation in liposomes, microparticles, microcapsules, and the like.

A therapeutic composition of the disclosure may comprise a carrier, such as one or more of a polymer, carbohydrate, peptide or derivative thereof, which may be directly or indirectly covalently attached to the compound. A carrier may be substituted with substituents described herein including without limitation one or more alkyl, amino, nitro, halogen, thiol, thioalkyl, sulfate, sulfonyl, sulfinyl, sulfoxide, hydroxyl groups. In aspects of the disclosure the carrier is an amino acid including alanine, glycine, praline, methionine, serine, threonine, asparagine, alanyl-alanyl, prolyl-methionyl, or glycyl-glycyl. A carrier can also include a molecule that targets a compound of the disclosure to a particular tissue or organ.

Compounds of the disclosure can be prepared using reactions and methods generally known to the person of ordinary skill in the art, having regard to that knowledge and the disclosure of this application including the Examples. The reactions are performed in solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the disclosure. It will also be recognized that another major consideration in the development of a synthetic route is the selection of the protecting group used for protection of the reactive functional groups present in the compounds described in this disclosure. An authoritative account describing the many alternatives to the skilled artisan is Greene and Wuts (Protective Groups In Organic Synthesis, Wiley and Sons, 1991).

A compound of the disclosure may be formulated into a pharmaceutical composition for administration to a subject by appropriate methods known in the art. Pharmaceutical compositions of the present disclosure or fractions thereof comprise suitable pharmaceutically acceptable carriers, excipients, and vehicles selected based on the intended form of administration, and consistent with conventional pharmaceutical practices. Suitable pharmaceutical carriers, excipients, and vehicles are described in the standard text, Remington: The Science and Practice of Pharmacy (21.sup.st Edition. 2005, University of the Sciences in Philadelphia (Editor), Mack Publishing Company), and in The United States Pharmacopeia: The National Formulary (USP 24 NF19) published in 1999. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the chug components may be combined with any oral, non-toxic, pharmaceutically, acceptable inert carrier such as ethanol, glycerol, water, and the like. Suitable binders (e.g., gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

The terms “administering” and “administration” as used herein refer to introducing a composition (e.g., haloperidol derivatives) of the present disclosure into a subject. As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia.

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, animals can be treated; the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g. gorilla or chimpanzee), and rodents such as rats and mice.

The term “pharmaceutically acceptable carrier” as used herein refers to a diluent, adjuvant, excipient, or vehicle with which a probe of the disclosure is administered, and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable, or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use. Pharmaceutically acceptable carriers may also include a dentifrice.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Additionally, the term “pharmaceutically acceptable” describes a material that is not biologically or otherwise undesirable, i.e., without causing an unacceptable level of undesirable biological effects or interacting in a deleterious manner.

As used herein, the terms “effective amount” and “amount effective” refer to an amount that is sufficient to achieve the desired result or to have an effect on an undesired condition or prevention of a disease or condition. The disease or condition can be an eye pathology such as macular degeneration (e.g., age-related macular degeneration). For example, a “therapeutically effective amount” refers to an amount that is sufficient to achieve the desired therapeutic result or to have an effect on undesired symptoms or prevention of a disease or condition of the subject to the desirable responses. The specific therapeutically effective dose level for any particular patient will depend upon a variety of factors including; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound (e.g., haloperidol derivatives) at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In an aspect, the dosage for haloperidol can be about 0.002 to 0.5 milligrams (mg), about 0.005 to 0.5 mg, about 0.001 mg, about 0.01 mg, about 0.025 mg, about 0.050 mg, about 0.075 mg, about 0.1 mg, about 0.125 mg, about 0.4 mg, or about 0.5 mg, while the molar equivalent of these amounts can be used as the dosage for the haloperidol derivatives.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof (e.g., eye pathology such as age-related macular degeneration). The effect can be therapeutic in terms of a partial or complete cure of a disease, condition, symptom, or adverse effect attributed to the disease, disorder, or condition. The term “treatment” as used herein can include any treatment of a disease, symptom, or condition in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions, (d) and/or tune the immune system of the subject. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition, and/or tuning the immune system of the subject to the desirable responses. Treating the disease, disorder, or condition can include ameliorating at least one symptom of the particular disease, disorder, or condition, even if the underlying pathophysiology is not affected.

As used herein, “therapeutic” can refer to treating, healing, and/or ameliorating a disease (e.g., eye pathology such as age-related macular degeneration), disorder, condition, or side effect, or to decreasing in the rate of advancement of a disease, disorder, condition, or side effect and/or tuning the immune system of the subject to the desirable responses for certain pathogens.

### Discussion

The present disclosure provides for methods of use of the haloperidol and haloperidol derivatives and their pharmaceutical compositions to treat eye pathologies. In an aspect, the eye pathology can be geographic atrophy, dry macular degeneration, wet macular degeneration, choroidal neovascularization, diabetic retinopathy, diabetic macular edema, retinal vein occlusion, neovascular glaucoma, retinal detachment, proliferative vitreoretinopathy, or glaucoma. The administration of the treatment can be via the eye. Additional details are provided in the Example.

Eye pathologies can include macular degeneration (e.g., age related macular degeneration), acute macular neuroretinopathy, choroidal neovascularization, macular edema (e.g., cystoid macular edema and diabetic macular edema), Behcet's disease, retinal disorders, diabetic retinopathy (e.g., proliferative diabetic retinopathy), retinal arterial occlusive disease, central retinal vein occlusion, uveitis (e.g., intermediate and anterior uveitis), glaucoma, neovascular glaucoma, retinal detachment, proliferative vitreoretinopathy and the like.

The term macular degeneration (MD) can also be referred to as: atrophic (dry) MD, exudative (wet) MD, age-related macular retinopathy (ARM), geographic atrophy, choroidal neovascularization, detached pigment retinal epithelium (PED), atrophy of pigment retinal epithelium (RPE). Macular degeneration (MD) also includes eye diseases irrelevant to age-related changes in a human organism, such as: vitelliform degeneration of Best, Stargardt disease, juvenile macular dystrophy, Behr's disease, Sorsby's dystrophy, Doyne honeycomb retinal dystrophy. Symptoms related to macular degeneration can include: drusen surrounded by white-yellow spots, submacular discoid scar of tissues, choroidal neovascularization, detached pigment retinal epithelium (PED), atrophy of pigment retinal epithelium (RPE), anomalous expansion of choroidal blood vessels, blurred or disturbed vision area, central dead point, pigment anomalies, mixed layer of thin granulation located on the inner side of Bruch's membrane, thickening and lowered permeability of Bruch's membrane.

The present disclosure provides for methods of treating eye pathologies, in particular macular degeneration. In an aspect, the method includes treating an eye pathology by administering to a subject in need thereof, a pharmaceutical composition. The pharmaceutical composition comprises a therapeutically effective amount of the compound or the pharmaceutical composition, where the compound has a formula represented by structure I or the pharmaceutically acceptable salt thereof below. In an aspect, administration can include an ocular administration. Ocular administration can include one of: topical administration, intraocular injection, drug eluting contact lens, implantation, electroporation, iontophoresis administration, intravitreal administration, intrastromal administration, intracameral administration, subTenon administration, subretinal administration, retrobulbar administration, peribulbar administration, suprachoroidal administration, choroidal administration, subchoroidal administration, conjunctival administration, subconjunctival administration, episcleral administration, posterior juxtascleral injection administration, or circumcorneal administration. In a particular aspect, ocular administration can be an ocular injection (e.g., microinjection). For ocular administration, the dosage for haloperidol can be about 0.002 to 0.5 milligrams (mg), about 0.005 to 0.5 mg, about 0.001 mg, about 0.01 mg, about 0.025 mg, about 0.050 mg, about 0.075 mg, about 0.1 mg, about 0.125 mg, about 0.4 mg, or about 0.5 mg, while the molar equivalent of these amounts can be used as the dosage for the haloperidol derivatives.

In one aspect, the compound can include haloperidol derivative compounds have a formula represented by structure I, or the pharmaceutically acceptable salt thereof:

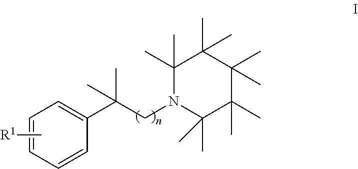

where each R¹ can be independently selected from hydrogen, halogen, and a C1 to C6 alkyl group; each R^{2a} and R^{2b} can be hydrogen or oxygen (when R^{2a} is oxygen, then there is a double bond between O and C and R^{2b} is not present); n can be an integer from 0 to 8; and R^{3a1}, R^{3a2}, R^{3b1}, R^{3b2}, R^{3c1}, R^{3c2}, R^{3d1}, R^{3d2}, R^{3e1} and R^{3e2} can be independently selected from hydrogen, halogen, a C1 to C6 alkyl group, and a substituted or unsubstituted aryl group. In some aspects, n can be an integer from 0 to 10. In other aspects, n can be an integer from 2 to 10. In some aspects, n can be an integer from 0 to 4 or 1 to 4. In other aspects, n can be an integer from 1 to 3. In further aspects, n is 3.

In some aspects, each R¹ can be independently selected from hydrogen, a halogen, and a C1 to C3 alkyl group. In further aspects, each R¹ can be independently selected from hydrogen a halogen, and methyl. In other aspects, one of R¹ can be a C1 to C6 alkyl group and the other of R¹ are hydrogen or a halogen. In further aspects, one of R¹ can be a C1 to C3 alkyl group and the other of R¹ are hydrogen or a halogen. In further aspects, one of R¹ is a methyl and the other of R¹ are hydrogen or a halogen.

In some aspects, one of R^{3a1}, R^{3a2}, R^{3b1}, R^{3b2}, R^{3c1}, R^{3c2}, R^{3d1}, R^{3d2}, R^{3e1} and R^{3e2} can be a substituted or unsubstituted aryl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or a halogen. In other aspects, one of R^{3a1}, R^{3a2}, R^{3b1}, R^{3b2}, R^{3c1}, R^{3c2}, R^{3d1}, R^{3d2}, R^{3e1} and R^{3e2} can be an aryl group substituted with at least one C1 to C6 alkyl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or a halogen. In other aspects, one of R^{3a1}, R^{3a2}, R^{3b1}, R^{3b2}, R^{3c1}, R^{3c2}, R^{3d1}, R^{3d2}, R^{3e1} and R^{3e2} can be a substituted or unsubstituted phenyl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or a halogen. In other aspects, one of R^{3a1}, R^{3a2}, R^{3b1}, R^{3b2}, R^{3c1}, R^{3c2}, R^{3d1}, R^{3d2}, R^{3e1} and R^{3e2} can be a phenyl group substituted with at least one C1 to C6 alkyl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or a halogen. In further aspects, one of R^{3a1}, R³², R^{3b1}, R^{3b2}, R^{3c1}, R^{3c2}, R^{3d1}, R^{3d2}, R^{3e1} and R^{3e2} can be a phenyl group substituted with at least one C1 to C3 alkyl group and the other of R^{3a1}, R^{3a2}, R^{3b1}, R^{3b2}, R^{3c1}, R^{3c2}, R^{3d1}, R^{3d2}, R^{3e1} and R^{3b2} are hydrogen or a halogen. In further aspects, one of R^{3a1}, R^{3a2}, R^{3b1}, R^{3b2}, R^{3c1}, R^{3c2}, R^{3d1}, R^{3d2}, R^{3e1} and R^{3e2} can be a phenyl group substituted with at least one methyl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or a halogen. In some aspects, R^{3a1}, R^{3a2}, R^{3b1}, R^{3b2}, R^{3c1}, R^{3c2}, R^{3d1}, R^{3d2}, R^{3e1} and R^{3e2} can be independently selected from hydrogen and one of the following:

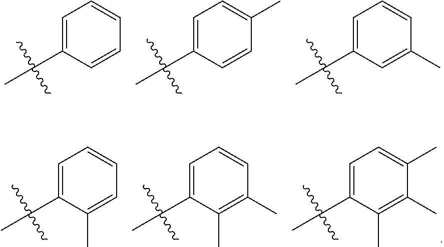

In other aspects, one of R^{3a}, R^{3b}, R^{3c}, R^{3d}, or R^{3e} can be one of the following:

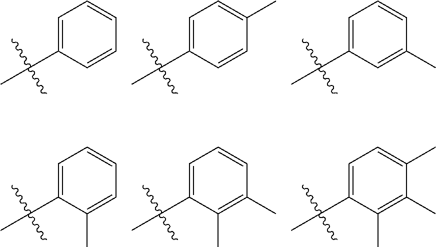

and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or a halogen.

In yet another aspect, method can use a pharmaceutical composition comprising a therapeutically effective amount of a compound to treat an eye pathology in a subject. In some aspects, the pharmaceutical composition can include a therapeutically effective amount of a haloperidol derivative compound to treat a condition (e.g., eye pathology) in a subject (e.g., animal or human subject). In some aspects, the pharmaceutical composition also includes a pharmaceutically acceptable carrier. In other aspects, the pharmaceutical composition is formulated for administering to a subject (e.g., human). In some aspects, the compound of the pharmaceutical composition has a formula represented by structure II or the pharmaceutically acceptable salt thereof:

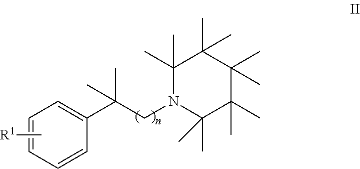

where each R¹ can be independently selected from hydrogen, halogen, and a C1 to C6 alkyl group; each R^{2a} and R^{2b} can be hydrogen or oxygen (when R^{2a} is oxygen, then there is a double bond between O and C and R^{2b} is not present); n can be an integer from 0 to 8; and R^{3a1}, R^{3a2}, R^{3b1}, R^{3b2}, R^{3c1}, R^{3c2}, R^{3d1}, R^{3d2}, R^{3e1} and R^{3e2} can be independently selected from hydrogen, halogen, a C1 to C6 alkyl group, and a substituted or unsubstituted aryl group. In some aspects, n can be an integer from 0 to 10. In other aspects, n can be an integer from 2 to 10. In some aspects, n can be an integer from 0 to 4. In other aspects, n can be an integer from 1 to 3. In further aspects, n is 3.

In some aspects, each R¹ can be independently selected from hydrogen, halogen, and a C1 to C3 alkyl group. In further aspects, each R¹ can be independently selected from hydrogen, halogen, and methyl. In other aspects, one of R¹ can be a C1 to C6 alkyl group and the other of R¹ are hydrogen or halogen. In further aspects, one of R¹ can be a C1 to C3 alkyl group and the other of R¹ are hydrogen or halogen. In further aspects, one of R¹ is a methyl and the other of R¹ are hydrogen or halogen.

In some aspects, one of R^{3a1}, R^{3a2}, R^{3b1}, R^{3b2}, R^{3c1}, R^{3c2}, R^{3d1}, R^{3d2}, R^{3e1} and R^{3e2} can be a substituted or unsubstituted aryl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or halogen. In other aspects, one of R^{3a1}, R^{3a2}, R^{3b1}, R^{3b2}, R^{3c1}, R^{3c2}, R^{3d1}, R^{3d2}, R^{3e1} and R^{3e2} can be an aryl group substituted with at least one C1 to C6 alkyl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or halogen. In other aspects, one of R^{3a1}, R^{3a2}, R^{3b1}, R^{3b2}, R^{3c1}, R^{3c2}, R^{3d1}, R^{3d2}, R^{3e1} and R^{3e2} can be a substituted or unsubstituted phenyl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or halogen. In other aspects, one of R^{3a1}, R^{3a2}, R^{3b1}, R^{3b2}, R^{3c1}, R^{3c2}, R^{3d1}, R^{3d2}, R^{3e1} and R^{3e2} can be a phenyl group substituted with at least one C1 to C6 alkyl group and the other of R^{3a1}, R^{3a2}, R^{3b1}, R^{3b2}, R^{3c1}, R^{3b2}, R^{3d1}, R^{3d2}, R^{3e1} and R^{3e2} are hydrogen or halogen. In further aspects, one of R^{3a1}, R^{3a2}, R^{3b1}, R^{3b2}, R^{3c1}, R^{3c2}, R^{3d1}, R^{3d2}, R^{3e1} and R^{3e2} can be a phenyl group substituted with at least one C1 to C3 alkyl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or halogen. In further aspects, one of R^{3a1}, R^{3a2}, R^{3b1}, R^{3b2}, R^{3c1}, R^{3c2}, R^{3d1}, R^{3d2}, R^{3e1} and R^{3e2} can be a phenyl group substituted with at least one methyl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or halogen. In some aspects, R^{3a1}, R^{3a2}, R^{3b1}, R^{3b2}, R^{3c1}, R^{3c2}, R^{3d1}, R^{3d2}, R^{3e1} and R^{3e2} can be independently selected from hydrogen and one of the following:

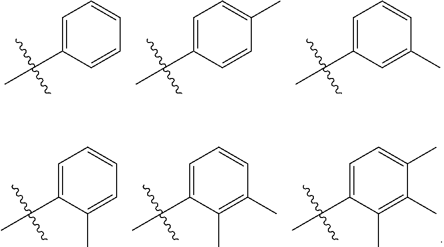

In other aspects, one of R^{3a}, R^{3b}, R^{3c}, R^{3d}, or R^{3e} can be one of the following:

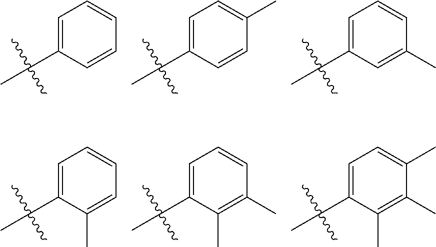

and the other of R^{3a1}, R^{3a2}, R^{3b1}, R^{3b2}, R^{3c1}, R^{3c2}, R^{3d1}, R^{3d2}, R^{3e1} and R^{3e2} are hydrogen or halogen.

In some aspects, the compound is not one of the following:

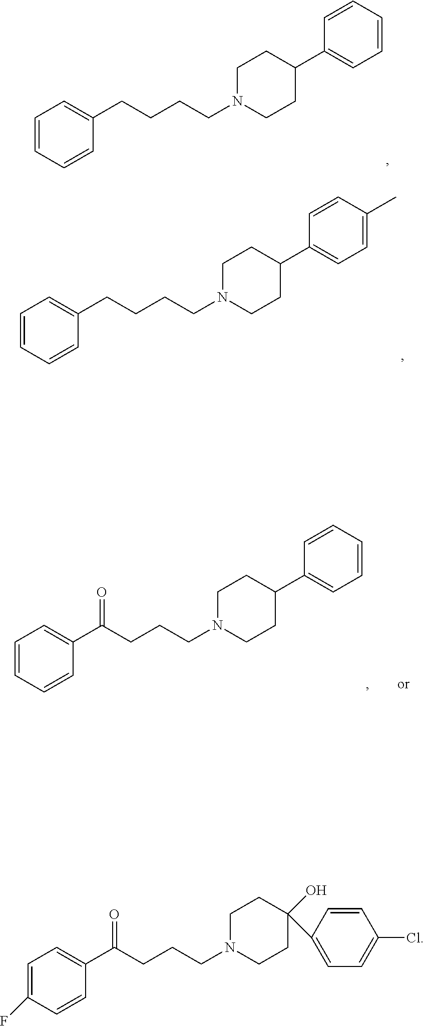 [SMILES: c1ccc(CCCCN2CCC(c3ccccc3)CC2)cc1 ; Cc1ccc(C2CCN(CCCCc3ccccc3)CC2)cc1 ; O=C(CCCN1CCC(c2ccccc2)CC1)c1ccccc1 ; O=C(CCCN1CCC(O)(c2ccc(Cl)cc2)CC1)c1ccc(F)cc1]

### Pharmaceutical Formulations and Routes of Administration

Embodiments of the present disclosure include the agent (e.g., haloperidol and haloperidol derivatives) as identified herein and can be formulated with one or more pharmaceutically acceptable excipients, diluents, carriers and/or adjuvants. In addition, embodiments of the present disclosure include the agent formulated with one or more pharmaceutically acceptable auxiliary substances. In particular the agent can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the agent can be administered to the subject using any means capable of resulting in the desired effect. Thus, the agent can be incorporated into a variety of formulations for therapeutic administration. For example, the agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agent may be administered in the form of its pharmaceutically acceptable salts, or a subject active composition may be used alone or in appropriate association, as well as in combination, with other pharmaceutically active compounds. The following methods and excipients are merely exemplary and are in no way limiting.

For oral preparations, the agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the agent can be utilized in aerosol formulation to be administered via inhalation. Embodiments of the agent can be formulated into pressurized acceptable propellants such as dichlorodifluoromethane, propane, nitrogen and the like.

Furthermore, embodiments of the agent can be made into suppositories by mixing with a variety of bases such as emulsifying bases or water-soluble bases. Embodiments of the agent can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the agent in a composition as a solution in sterile water, normal saline or another pharmaceutically acceptable carrier.

Embodiments of the agent can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient (triamino-pyridine derivative and/or the labeled triamino-pyridine derivative) encapsulated in liposome vehicles in accordance with the present disclosure.

In an embodiment, the agent can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the agent can be accomplished using any of a variety of refillable, pump systems. Pumps provide consistent, controlled release over time. In some embodiments, the agent can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, the agent can be delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the agent are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the agent adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) can be delivered in a controlled release system. For example, the agent may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) include those formed by impregnation of the agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### Dosages

Embodiments of the agent (e.g., haloperidol and haloperidol derivatives) can be administered to a subject in one or more doses. Those of skill will readily appreciate that dose levels can vary as a function of the specific the agent administered, the severity of the symptoms and the susceptibility of the subject to side effects. Preferred dosages for a given compound are readily determinable by those of skill in the art by a variety of means.

In an embodiment, multiple doses of the agent are administered. The frequency of administration of the agent can vary depending on any of a variety of factors, e.g., severity of the symptoms, and the like. For example, in an embodiment, the agent can be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). As discussed above, in an embodiment, the agent is administered continuously.

The duration of administration of the agent, e.g., the period of time over the agent is administered, can vary, depending on any of a variety of factors, e.g., patient response, etc. For example, the agent in combination or separately, can be administered over a period of time of about one day to one week, about two weeks to four weeks, about one month to two months, about two months to four months, about four months to six months, about six months to eight months, about eight months to 1 year, about 1 year to 2 years, or about 2 years to 4 years, or more.

In an aspect, the dosage for haloperidol can be about 0.002 to 0.5 milligrams (mg), about 0.005 to 0.5 mg, about 0.001 mg, about 0.01 mg, about 0.025 mg, about 0.050 mg, about 0.075 mg, about 0.1 mg, about 0.125 mg, about 0.4 mg, or about 0.5 mg. In an aspect, the dosage for haloperidol derivatives can be about 0.002 to 0.5 milligrams (mg), about 0.005 to 0.5 mg, about 0.001 mg, about 0.01 mg, about 0.025 mg, about 0.050 mg, about 0.075 mg, about 0.1 mg, about 0.125 mg, about 0.4 mg, or about 0.5 mg, or molar equivalent of these amounts as compared to haloperidol. In an aspect, administration can include those described herein and in particular systemic administration (e.g., IV, PO, IM, SC, INH PR).

For ocular administration, the dosage for haloperidol can be about 0.002 to 0.5 milligrams (mg), about 0.005 to 0.5 mg, about 0.001 mg, about 0.01 mg, about 0.025 mg, about 0.050 mg, about 0.075 mg, about 0.1 mg, about 0.125 mg, about 0.4 mg, or about 0.5 mg, while the molar equivalent of these amounts can be used as the dosage for the haloperidol derivatives.

### Routes of Administration

Embodiments of the present disclosure provide methods and compositions for the administration of the agent (e.g., haloperidol and haloperidol derivatives) to a subject (e.g., a human) using any available method and route suitable for drug delivery, including in vivo and ex vivo methods, as well as systemic and localized routes of administration.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An agent can be administered in a single dose or in multiple doses.

Embodiments of the agent can be administered to a subject using available conventional methods and routes suitable for delivery of conventional drugs, including systemic or localized routes. In general, routes of administration contemplated by the disclosure include, but are not limited to, enteral, parenteral, or inhalational routes.

In an aspect, the method includes ocular administration of the haloperidol derivatives. An ocular route of administration can include topical (e.g., drops, gels, ointment, emulsions, suspensions, and the like), injections (e.g., microinjection with a microneedle), drug eluding contact lenses, implantation, electroporation, iontophoresis, intravitreal, intrastromal, intracameral, subtenon, subretinal administration, retrobulbar, peribulbar, suprachoroidal, choroidal, subchoroidal, conjunctival, subconjunctival, episcleral, posterior juxtascleral injection, periocular, intracameral, circumcorneal, and the like. The compositions may be administered using one or more of the routes described herein to the, for example, vitreous, aqueous humor, sclera, conjunctiva, the area between the sclera and conjunctiva, the retina choroids tissues, macula, or other area in or proximate to the eye of an individual.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the agent. Where systemic delivery is desired, administration typically involves invasive or systemically absorbed topical or mucosal administration of pharmaceutical preparations.

In an embodiment, the agent can also be delivered to the subject by enteral administration. Enteral routes of administration include, but are not limited to, oral and rectal (e.g., using a suppository) delivery.

Methods of administration of the agent through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### Example 1

Genomic association studies have identified multiple pathways that might be affected in AMD. However, multiple drugs that block these pathways have failed in clinical trials in geographic atrophy over the past decade. Therefore, drugs were identified using a different approach. A health insurance administrative claims database was analyzed to identify existing FDA-approved drugs that are associated with a reduced risk of developing dry AMD. By using the power of these very large patient datasets, the repurposing of an existing approved drug for a new disease indication was enabled. In this study, exposure to haloperidol, an FDA-approved drug for the treatment of schizophrenia or Tourette's disorder, was examined for risk of developing dry AMD. It was also studied whether haloperidol inhibits activation of the inflammasome, a inflammatory pathway that promotes dry AMD (Tarallo, 2012). Finally, it was studied whether haloperidol inhibits RPE degeneration in an animal model of dry AMD.

### Methods

### Health Insurance Claims Databases Analyses

Health insurance database information contains de-identified data that are HIPAA-compliant and were deemed by the University of Virginia Institutional Review Board (IRB) as exempt from IRB approval requirements.

### Data Source

The retrospective study used claims data from the PearlDiver Mariner database, which contains data on health care claims and medication usage for persons in provider networks over the time period 2010 to the second quarter of 2018.

### Sample Selection

Patients were included in the analysis if they had continuous enrollment in the plan for at least 1 year and were at least 50 years of age at baseline. Individuals with pre-existing dry AMD (≥1 medical claims prior to diagnosis of schizophrenia or Tourette's disorder) were excluded. Disease claims were identified by International Classification of Diseases (ICD)-9-CM and ICD-10-CM codes.

### Independent Variable

Exposure to haloperidol—the independent variable—was determined by whether patients filled ≥1 outpatient pharmacy prescriptions for generic or brand versions, either in sole form or as a combination medication, as identified by National Drug Codes.

### Dependent Variable

Time to initial diagnosis of dry AMD was the dependent variable.

### Analyses

Analyses were performed with the use of R software, version 4.2.2 (the R project (www.r-project.org)). To analyze the risk of dry AMD between haloperidol users and haloperidol non-users, an adjusted Cox proportional hazards regression analysis was performed, and the hazard ratio was analyzed by the likelihood ratio test. The restricted maximum-likelihood estimator method was used to estimate the between-study variance. This adjusted model included confounding covariates that influence dry AMD risk: age, sex, smoking, body mass index, and Charlson comorbidity index. Kaplan-Meier survival plots were analyzed by log rank test. Statistical tests were 2-sided. P values <0.05 were considered statistically significant.

### Propensity Score Matching

To evaluate the robustness of the findings and mitigate any possible residual confounding, we estimated propensity-score models including use of haloperidol and no use of haloperidol. The individual propensities for starting haloperidol treatment were estimated with the use of logistic regression. As predictors, the propensity-score models included the set of variables which displayed P values <0.1 in logistic regression analyses. We used the R package MatchIt to perform matching in a 1:1 ratio using greedy nearest neighbor matching. In addition, to control for any residual covariate imbalance, we estimated the relative hazard in the propensity score-matched groups using the multivariable Cox models that included the covariates from the multivariable regression analysis employed for the original unmatched group analyses. Statistical tests were two-sided. P values <0.05 were considered statistically significant.

### ELISA

Human THP-1 cells were stimulated with LPS (125 ng/ml) for 4 h and ATP (5 mM) for 30 min. For all experiments, cells were pretreated with haloperidol (1, 10, or 100 μM) for 1 h and again after the activating stimuli. Secreted human IL-β in the conditioned cell culture media was detected using an ELISA kit (R&D Systems) according to the manufacturer's instructions. Mean and SEM values are presented. Student's t test was used to determine whether haloperidol-treatment reduced IL-1β levels compared to LPS- and ATP-treated cells.

### Induction of RPE Degeneration and Drug Treatments

C57BL/6J mice (The Jackson Laboratory) were anesthetized with ketamine hydrochloride and xylazine. In vitro-transcribed Alu RNA (300 ng in 1 μL) was injected subretinally to induce RPE degeneration (Kaneko, 2011). Various doses of haloperidol (Sigma Aldrich; 1-500 ng in 0.5 μL) or PBS (vehicle) was injected into the vitreous humor 24 h before and immediately after Alu RNA injection. Alternatively, various doses of haloperidol (2-10 μg) were administered via intraperitoneal administration on days 1 through 6 after Alu RNA injection. Animals were euthanized 7 days after Alu RNA injection, and the eyes were enucleated.

### Assessment of RPE Degeneration

RPE health was assessed by immunofluorescence staining of zonula occludens-1 (ZO-1) on RPE flat mounts. Flat mounts were fixed with 2% PFA, stained with rabbit polyclonal antibodies against mouse ZO-1 (Invitrogen), visualized with Alexa Fluor 594 (Invitrogen), and imaged (A1R Nikon). Images were graded as healthy or degenerated in masked fashion as previously described (Kerur, 2018). Proportions of eyes with degeneration were compared using Fisher's exact test.

### Results

Haloperidol Use Associated with Reduced Development of Dry AMD

We performed a retrospective, longitudinal cohort analysis in the PearlDiver Mariner database (comprising 150 million Americans from 2010 to 2018) among patients with schizophrenia or Tourette's disorder (the population that would be exposed to haloperidol or other anti-psychotics) aged 50 or older (the population at risk) to assess the risk of dry AMD.

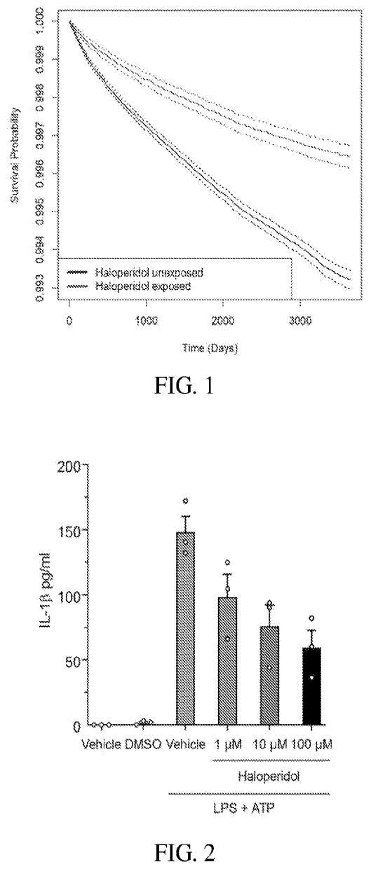

First, we performed Kaplan-Meier survival analyses to estimate the probability of developing dry AMD: haloperidol use was associated with a significantly slower rate of developing dry AMD in the Mariner database (P<0.001) (FIG. 1).

Next, we performed Cox proportional hazards regression analyses to estimate the hazard of dry AMD in relation to haloperidol use. Patients in these databases were not randomly assigned to haloperidol treatment; therefore, we performed propensity score matching, a causal inference approach (Rosenbaum & Rubin, 1983), to assemble cohorts with similar baseline characteristics, thereby reducing possible bias in estimating treatment effects. Additionally, to control for any residual covariate imbalance, we adjusted for confounders associated with dry AMD: age, sex, smoking, and body mass index, and Charlson comorbidity index, a measure of overall health. These adjusted Cox proportional hazards regression models in the propensity-score-matched populations also showed a protective association of haloperidol use (Table 1). In Mariner, haloperidol exposure was associated with a 25% reduced hazard of developing dry AMD (adjusted hazard ratio: aHR, 0.747; 95% CI, 0.670 to 0.833; P<0.001).

TABLE 1

| | | Lower 95% | Upper 95% | |
|---|---|---|---|---|
| | Hazard | confidence | confidence | |
| | Ratio | interval | interval | P value |
| Haloperidol use | 0.7468 | 0.6695 | 0.8330 | 1.6 × 10⁻⁷ |
| Age | 1.1079 | 1.1020 | 1.1138 | <2 × 10⁻¹⁶ |
| Male sex | 0.7602 | 0.6762 | 0.8545 | 4.4E−06 |
| Smoking | 0.8152 | 0.7187 | 0.9246 | 0.00147 |
| Body Mass Index: | 1.2006 | 1.0422 | 1.3831 | 0.01134 |
| 30-40 | | | | |
| Charlson | 1.0668 | 1.0462 | 1.0878 | 8.2 × 10⁻¹¹ |
| comorbidity index | | | | |

Concordance = 0.857 (se = 0.005)
Likelihood ratio test = 2542 on 6 df, p < 2 × 10⁻¹⁶
Wald test = 1973 on 6 df, p < 2 × 10⁻¹⁶
Score (log-rank) test = 2460 on 6 df, p < 2 × 10⁻¹⁶

### Haldol Inhibits Inflammasome Activation

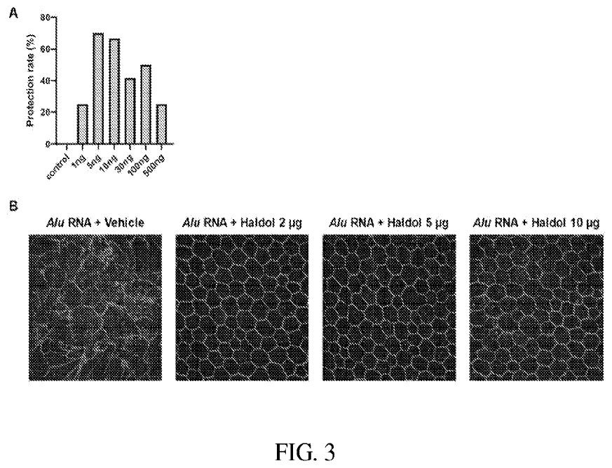

Haloperidol was tested for inhibition of inflammasome activation. Inflammasome activation was assessed in human THP-1 monocytes by measuring IL-1ß secretion via ELISA. IL-1β release was robustly induced by LPS and ATP stimulation, and it was reduced by exposure to haloperidol (P<0.05 for each of the haloperidol-treated groups compared to vehicle-treated group, following LPS+ATP stimulation, Student's t test) (FIG. 2). These data demonstrate that haloperidol inhibits inflammasome activation.

### Haldol Inhibits RPE Degeneration

Alu RNA, a retrotransposon-derived transcript that is increased in abundance in the RPE of human eyes with geographic atrophy, induces inflammasome activation and RPE degeneration (Kaneko, 2011). We tested the effect of haloperidol on Alu RNA-induced RPE degeneration in mice. We tested haloperidol administered via local intravitreous injection or via systemic intraperitoneal administration. We tested doses ranging from 1 ng to 500 ng injected into the vitreous humor. This dose-ranging study showed dose-dependency with a maximal protective effect at 5 ng of haloperidol (FIG. 3A). At this dose, 70% of eyes were protected against RPE degeneration. We also tested the effects of systemically administered haloperidol. Daily intraperitoneal injection of haloperidol at 2 μg, 5 μg, or 10 μg, all induced 70-80% protection against RPE degeneration (FIG. 3B). These data demonstrate a powerful inhibitory effect of haloperidol in a human disease-relevant animal model of geographic atrophy.

### Discussion

These studies provide evidence that haloperidol inhibits both inflammasome activation. This, combined with the finding that oral haloperidol use is associated with a reduced risk of developing dry AMD in people, supports the concept that oral administration of haloperidol in humans can achieve levels capable of exerting biological activity against dry AMD. It is important to note that patients taking haloperidol have a serum concentration exceeding 20 ng/ml (Coryell, 1998). Accounting for interspecies allometric conversion (Nair & Jacob, 2016), we found that lower dose was sufficient to protect against RPE degeneration. Therefore, doses of haloperidol lower than those currently prescribed for psychiatric disorders can be beneficial for dry AMD.

A strength of our health insurance database analyses is that findings were adjusted for confounders and performed propensity score matching, which increases the internal validity of our conclusion. We also provide evidence that intraocular or systemic administration of haloperidol prevents RPE degeneration in an animal model. As haloperidol is a cell-permeable small molecule, another mode of delivery is a sustained release intraocular implant, a drug delivery modality that has been used effectively for other ocular indications.

Despite numerous advances into the mechanisms of dry AMD, there is still no approved therapy for this disease. Traditional approaches to drug development can be expensive and time consuming: on average, a new FDA-approved drug takes more than a decade and costs $3 billion (present-day dollars) to develop (Dimasi, 2016). Our identification of the unrecognized therapeutic activity of an existing FDA-approved drug using big data mining, coupled with demonstrating its efficacy in a disease-relevant model, can greatly accelerate and reduce the cost of drug development.

### REFERENCES

1. Ambati, J. et al. Age-related macular degeneration: Etiology, pathogenesis, and therapeutic strategies. Surv Ophthalmol. 2003; 48:257-293.
2. Coryell, W. et al. Haloperidol Plasma Levels and Dose Optimization. Am J Psychiatry 1998; 155:48-53
3. DiMasi, J. A. et al. Innovation in the pharmaceutical industry: New estimates of R&D costs. J. Health Econ. 2016; 47:20-33.
4. Kaneko H., et al. DICER1 deficit induces Alu RNA toxicity in age-related macular degeneration. Nature 2011; 471:325-30.
5. Kerur N., et al. cGAS drives non-canonical NLRP3 inflammasome in age-related macular degeneration. Nat Med. 2018; 24:50-61.
6. Nair, A. B. & Jacob, S. A simple practice guide for dose conversion between animals and human. J Basic Clin Pharm. 2016; 7:27-31.
7. Rosenbaum P. R. and Rubin, D. B. The central role of the propensity score in observational studies for causal effects. Biometrika 1983; 70:41-55.
8. Tarallo, V. et al. DICER1 loss and Alu RNA induce age-related macular degeneration via the NLRP3 inflammasome and MyD88. Cell 2012; 149:847-859.
9. Wong W. L. et al. Global prevalence of age-related macular degeneration and disease burden projection for 2020 and 2040: A systematic review and meta-analysis. Lancet Glob. Health 2014; 2: e106-e116.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 percent to about 5 percent” should be interpreted to include not only the explicitly recited concentration of about 0.1 weight percent to about 5 weight percent but also include individual concentrations (e.g., 1 percent, 2 percent, 3 percent, and 4 percent) and the sub-ranges (e.g., 0.5 percent, 1.1 percent, 2.2 percent, 3.3 percent, and 4.4 percent) within the indicated range. The term “about” can include traditional rounding according to significant figures of the numerical value. In addition, the phrase “about ‘x’ to ‘y’” includes “about ‘x’ to about ‘y’”.

Many variations and modifications may be made to the above-described aspects. All such modifications and variations are intended to be included herein within the scope of this disclosure and protected by the following claims.

1. A method for treating an eye pathology comprising: administering to a subject in need thereof, a pharmaceutical composition, wherein the pharmaceutical composition comprises a therapeutically effective amount of the compound or the pharmaceutical composition, wherein the compound has a formula represented by structure I or the pharmaceutically acceptable salt thereof: 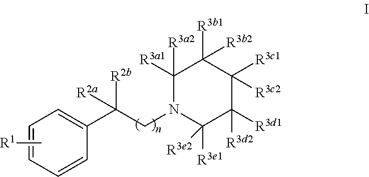
wherein
each R¹ is independently selected from hydrogen, halogen, and a C1 to C6 alkyl group;
R^{2a} and R^{2b} are each independently selected from hydrogen or oxygen, when R^{2a} is oxygen, oxygen has a double bond to the carbon atom and the carbon atom is not bonded to R^{2b} due to the double bond to carbon;
n is an integer from 0 to 8; and
R^{3a1}, R^{3a2}, R^{3b1}, R^{3b2}, R^{3c1}, R^{3c2}, R^{3d1}, R^{3d2}, R^{3e1} and R^{3e2} are independently selected from hydrogen, halogen, —OH, a C1 to C6 alkyl group, and a substituted or unsubstituted aryl group.

2. (canceled)

3. The method of claim 1, wherein the eye pathology is geographic atrophy.

4. The method of claim 1, wherein the eye pathology is dry macular degeneration.

5. The method of claim 1, wherein the eye pathology is wet macular degeneration.

6. The method of claim 1, wherein the eye pathology is choroidal neovascularization.

7. The method of claim 1, wherein the eye pathology is diabetic retinopathy.

8. The method of claim 1, wherein the eye pathology is diabetic macular edema.

9. The method of claim 1, wherein the eye pathology is retinal vein occlusion.

10. The method of claim 1, wherein the eye pathology is neovascular glaucoma.

11. The method of claim 1, wherein the eye pathology is retinal detachment.

12. The method of claim 1, wherein the eye pathology is proliferative vitreoretinopathy.

13. The method of claim 1, wherein the eye pathology is glaucoma.

14. The method of claim 1, wherein administration is an ocular administration.

15. (canceled)

16. The method of claim 14, wherein ocular administration is selected from one of: intravitreous administration, suprachoroidal administration, sub-Tenon administration, periocular administration, subretinal administration, intracameral administration, or topical administration.

17. (canceled)

18. The method of claim 1, wherein n is 4, wherein each R¹ is independently selected from hydrogen and methyl.

19. (canceled)

20. The method of claim 1, wherein one of R¹ is a methyl and the other of R¹ are hydrogen.

21. The method of claim 1, wherein n is an integer from 1 to 8, wherein one of R^{3a1}, R^{3a2}, R^{3b1}, R^{3b2}, R^{3c1}, R^{3c2}, R^{3d1}, R^{3d2}, R^{3e1} and R^{3e2} is a substituted or unsubstituted aryl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen.

22. The method of claim 1, wherein n is an integer from 1 to 8, wherein one of R^{3a1}, R^{3a2}, R^{3b1}, R^{3b2}, R^{3c1}, R^{3c2}, R^{3d1}, R^{3d2}, R^{3e1} and R^{3e2} is a substituted or unsubstituted phenyl group and the other of R^{3a1}, R^{3a2}, R^{3b1}, R^{3b2}, R^{3c1}, R^{3c2}, R^{3d1}, R^{3d2}, R^{3e1} and R^{3e2} are hydrogen.

23. The method of claim 1, wherein n is an integer from 1 to 8, wherein each of R^{3a1}, R^{3a2}, R^{3b1}, R^{3b2}, R^{3c1}, R^{3c2}, R^{3d1}, R^{3d2}, R^{3e1} and R^{3e2} are independently selected from hydrogen and one of the following:

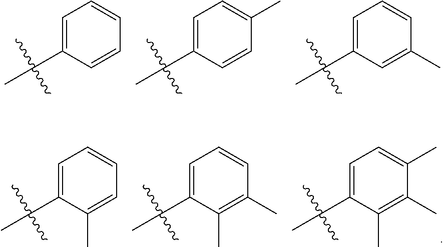

24. The method of claim 1, wherein n is an integer from 1 to 8, wherein one of R^{3a1}, R^{3a2}, R^{3b1}, R^{3b2}, R^{3c1}, R^{3c2}, R^{3d1}, R^{3d2}, R^{3e1} and R^{3e2} is one of the following: 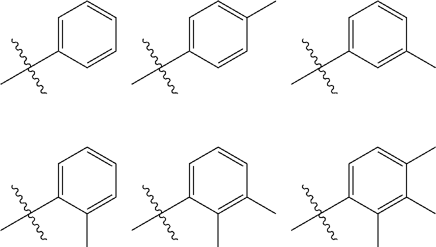
and the other of R^{3a1}, R^{3a2}, R^{3b1}, R^{3b2}, R^{3c1}, R^{3c2}, R^{3d1}, R^{3d2}, R^{3e1} and R^{3e2} are hydrogen.

25-26. (canceled)
